(19) United States
(12) Patent Application Publication
(10) Pub. No.: US 2022/0248948 A1
(43) Pub. Date: Aug. 11, 2022

(54) VISUAL FIELD ASSESSMENT SYSTEM AND METHOD

(71) Applicant: The University of Manchester, Manchester Greater Manchester (GB)

(72) Inventor: Tariq Aslam, Manchester Greater Manchester (GB)

(21) Appl. No.: 17/606,310

(22) PCT Filed: Apr. 29, 2020
(86) PCT No.: PCT/GB2020/051043

§ 371 Date: Oct. 25, 2021

(30) Foreign Application Priority Data
Apr. 29, 2019 (GB) 1905934.4

(51) Int. Cl. A61B 3/024; G06F 3/01; G06T 13/00; G06F 3/14; G06T 11/00; A61B 3/00; A63F 13/537

(52) U.S. Cl. CPC A61B 3/024; G06F 3/013; G06T 13/00; A63F 13/537; G06T 11/00; A61B 3/0041; G06F 3/14

(57) ABSTRACT

A visual field assessment system (100) comprises a display device (116) having a display area; a user input device (118), and a processor (102, 104). The processor is configured to intermittently display a first visual stimulus (306) in a central display region (301 A) of the display area and intermittently display a second visual stimulus (308) in a peripheral display region (301 B) of the display area. The processor determines whether the user has correctly visually detected the second visual stimulus based on use of the user input device, and selectively displays a graphical object (310) moving from the peripheral display region towards the central display region based on the determination.

### FIELD OF THE INVENTION

The present invention relates to visual field assessment systems and methods.

### BACKGROUND TO THE INVENTION

A basic premise of visual field assessment is to be able to assess if a person can see objects not in the centre, but in the periphery, of his/her vision. To do this it is desirable to make sure they are looking at a certain focus point and ask them to respond to peripheral visual stimuli that appear at a distance from the central focus point. They must keep looking at the focus point, and not at the peripheral stimuli, to make sure it is indeed their peripheral vision, and not central vision, that is being tested. The peripheral locations need to be at various angles measured from the focus point.

However, it is difficult to get children (and some adults) to keep looking at a focus point when peripheral objects appear—they tend to shift their gaze to the peripheral point, which reduces the effectiveness and result quality of peripheral vision testing.

### SUMMARY OF THE INVENTION

Embodiments of the present invention can address the problems discussed above and provide improved visual field assessment results. Embodiments can address the challenge of getting users, in particular children, to look in the direction of a central display region only (where first visual stimuli are intermittently displayed), and yet still respond to other visual stimuli which are intermittently displayed in a peripheral display region.

According to a first aspect, there is provided a visual field assessment system comprising:
a display device having a display area;
a user input device, and
a processor configured to:
   intermittently display a first visual stimulus in a central display region of the display area;
   intermittently display a second visual stimulus in a peripheral display region of the display area;
   determine whether the user has correctly visually detected the second visual stimulus based on use of the user input device, and
   selectively display a graphical object moving from the peripheral display region towards the central display region based on the determination.

The processor may determine that the user has correctly visually detected the second visual stimulus if the use of the user input device indicates that the user activated the user input device within a time frame related to the intermittent display of the second visual stimulus, e.g. the user input device is activated within a predetermined time period (e.g. number of microseconds) following the display of the second visual stimulus (or within a predetermined time period after the second visual stimulus has disappeared/stopped being displayed).

The processor may selectively display the graphical object moving from the peripheral display region towards the central display region as an animated display. The second visual stimulus may comprise intermittently displaying the graphical object in the peripheral display region.

The processor may be configured to intermittently display a plurality of the second visual stimuli at different locations within the peripheral display region. The processor may be configured to:
determine that the user has correctly visually detected at least one of the plurality of second visual stimuli based on use of the user input device;
intermittently display a first further one of the plurality of second visual stimuli at a first location within the peripheral display region;
determine whether the user has correctly visually detected the first further one of the plurality of second visual stimuli based on use of the user input device;
intermittently display a second further one of the plurality of second visual stimuli at a second location within the peripheral display region;
determine whether the user has correctly visually detected the second further one of the plurality of second visual stimuli based on use of the user input device, and
if the processor determines that the user has not correctly visually detected the first further one and the second further one of the plurality of second visual stimuli then the processor is configured to display a modified second visual stimulus.

The modified second visual stimulus may be visually different to the second visual stimulus, e.g. may be a different size (typically larger), a different shape, a different colour, etc. The modified second visual stimulus may be displayed at the first location of the peripheral display region. The processor may be configured to determine whether the user has correctly visually detected the modified second visual stimuli based on use of the user input device. If the processor determines that the user has not correctly visually detected the modified second visual stimuli then the processor may be configured to display the modified second visual stimuli moving from the peripheral display region towards the central display.

The processor may be configured to subsequently re-display the second further one of the plurality of second visual stimuli at the second location within the peripheral display region, and determine whether the user has correctly visually detected the second further one of the plurality of second visual stimuli based on use of the user input device.

The system may further comprise an eye/gaze tracking device. The processor may be configured to use the eye/gaze tracking device to determine if a gaze of the user is directed at the central display region during the intermittent display of the second visual stimulus.

The processor may be configured to intermittently display a plurality of the first visual stimuli at a plurality of different locations within the central display region. For example, positions of the first visual stimuli may move, e.g. by being displayed at various locations within the central display region.

The processor may be configured to determine whether a user has correctly visually detected the first visual stimulus based on use of a user input device.

The processor may be configured to generate an output indicating a performance of the user in visually detecting at least the second visual stimuli. The output may include: number of modified second visual stimuli displayed; number of modified second visual stimuli missed by the user; number of first visual stimuli missed. The second visual stimulus may be displayed during a time window when the first visual stimulus is not displayed. The time window may be adjusted during the gameplay, e.g. based on a user's performance.

The processor may be configured to provide a positive consequence (e.g. in terms of game rules) if the user has correctly visually detected the first visual stimulus and/or the second visual stimulus. The processor may be configured to provide a negative consequence (e.g. in terms of game rules) if the user has not correctly visually detected the first visual stimulus and/or the second visual stimulus.

The system may further include a panel that, in use, is positioned/positionable between the user and at least one of the display device and the user input device. The panel may comprise a surface of a housing containing at least one of the display device and the user input device.

The processor may be configured to generate a display in the central display region such that a gaze of the user is focused/maintained on the central display region (even whilst the second visual stimulus is intermittently displayed in the peripheral display region).

According to another aspect, there is provided a (computer-implemented) visual field assessment method comprising:
intermittently displaying a first visual stimulus in a central display region of a display area of a display device;
intermittently displaying a second visual stimulus in a peripheral display region of the display area;
determining whether a user has correctly visually detected the second visual stimulus based on use of a user input device, and
selectively displaying a graphical object moving from the peripheral display region towards the central display region based on the determination.

According to a further aspect there is provided a computer readable medium storing a computer program to operate visual field assessment methods substantially as described herein.

### BRIEF DESCRIPTION OF THE DRAWINGS

For a better understanding of the invention, and to show how exemplary embodiments of the same may be brought into effect, reference will be made, by way of example only, to the accompanying diagrammatic Figures, in which:

FIG. 1 is a schematic illustration of a system according to an embodiment;

FIG. 2 shows a specific example of the system;

FIGS. 3A-3D show examples of graphical displays generated by the system, and

FIG. 4 shows an example output generated by the system.

### DETAILED DESCRIPTION OF THE INVENTION

According to the present invention there is provided a system and method as set forth in the appended claims. Other features of the invention will be apparent from the dependent claims, and the description that follows.

The optional features set out herein may be used either individually or in combination with each other where appropriate and particularly in the combinations as set out in the accompanying claims. The optional features for each aspect or exemplary embodiment of the invention, as set out herein are also applicable to all other aspects or exemplary embodiments of the invention, where appropriate. In other words, the skilled person reading this specification should consider the optional features for each aspect or exemplary embodiment of the invention as interchangeable and combinable between different aspects and exemplary embodiments.

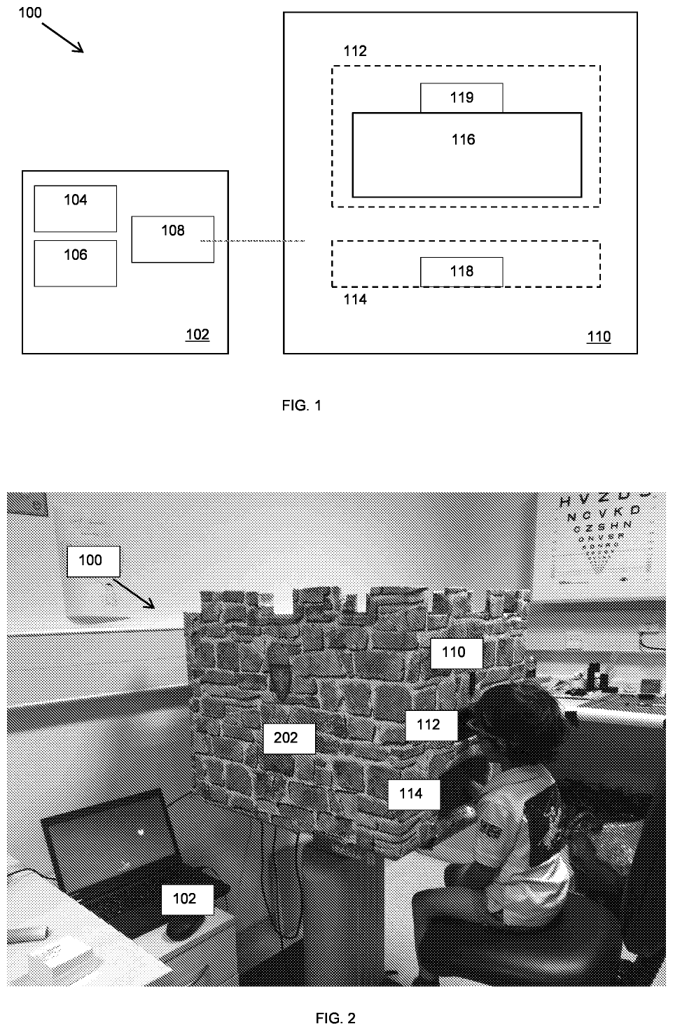

FIG. 1 schematically illustrates an example visual field assessment system 100. The system can comprise a conventional electronic computer 102, which can have well-known components, such as a processor 104 and memory 106, as well as other features, such as at least one communications interface 108, etc. The computer may comprise a laptop PC, a desktop PC, a tablet computer, or a remote server, etc.

Some embodiments of the system 100 can further include a panel 110 that, in use, is positioned between the user and at least one component of the system. Typically, the user will sit or stand in front of the panel as viewed in FIG. 1. In some embodiments the panel comprises a flat surface that may be part of a housing, e.g. a generally cuboid shape. However, it will be appreciated that the design and dimensions of the panel/housing can vary and that it can be formed of any suitable material(s), e.g. a rigid or semi-flexible material, such as plastic, cardboard, etc. In use, the user can place their head, possibly on an adjustable head/chin rest, to view inside the housing. In some cases, material, e.g. black felt, may be used to cover sides of the system.

In some embodiments the panel 110 includes at least one aperture so that the user can see and/or physically manipulate the component(s) of the system 100 that are positioned on the other side of it. In the illustrated example the panel includes a first, upper aperture 112 and a second, lower aperture 114. In the illustrated example, a display device 116 can be at least partially viewed through the upper aperture, and a user input device 118 can be physically accessible through the lower aperture. The use of the panel/housing can help focus the user's gaze on the display device, as well as help mentally immerse him/her in the game/assessment.

The display device 116 and the user input device 118 can be in communication with the computer 112 via its interface(s) 108, which can comprise any suitable wired or wireless interface(s), e.g. HMDI, serial cable, Bluetooth™, a network link including Wifi™ or the internet, etc. The display device can comprise any suitable display technology, such as an LED or LCD screen. The user input device can comprise any suitable device, such as a push-button, joystick, etc. Typically, the user input device comprises a single response button to respond to both central and peripheral visual stimuli (detailed below). After extensive testing this was found to be a necessary simplification for most children.

In some embodiments the system 100 can further include an eye/gaze tracking device 119. This will typically be located on/adjacent the display device 116 and is also in communication with the computer 102.

In some embodiments, the visual field assessment method includes gaming elements. In the main detailed embodiment described herein the game has a theme that a queen has left home to go to the shops and left her son, the prince Caspar, in a castle alone. He has left a door open and allowed a whole lot of creatures—called ‘googlies’—into the castle. An aim of the game is for him to sweep/vacuum them up before his mum gets back. The prince appears as the central/main game with a brush to sweep up central googlies that appear in the central display region into a receptacle. He also has a vacuum to suck in any peripheral googlies that appear in the peripheral display region. It will be understood that this is exemplary only and many variations are possible—both in the graphical/visual presentation of the method and the game-type rules that may be applied.

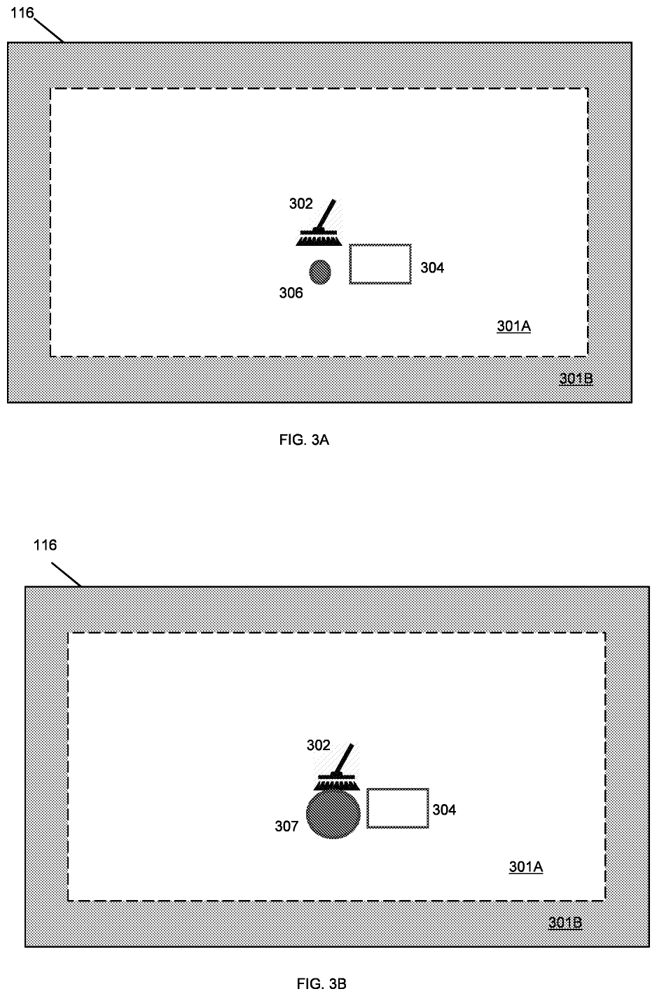

FIG. 2 shows a specific embodiment of the system 100, where the panel 110 is part of a housing 202 that represents a castle type building in order to tie in with visual elements of an example of the visual field assessment game. However, it will be understood that this is merely exemplary and many other variants are possible. For example, all components of the computer may be located behind the panel 110; the panel may only be located between the user and the display device 116 with other components of the system being located below, or the side or it; there may be no panel at all with the display and user input devices being free-standing; there may be more than one display device (e.g. one central useable as the central display region and at least one peripheral display device useable as the peripheral display region(s)), and so on.

Some embodiments can solve the problems discussed above by using gamification of visual field assessment. Some embodiments can have two aspects of a visual field assessment running in parallel: a central game producing first visual stimuli on which a person is required to maintain focus, and a peripheral game where peripheral visual stimuli are displayed so that the person also has to respond to these whilst still looking directly at the central visual stimuli. Typically, the location of the central visual stimuli/game moves, e.g. by being displayed at various locations on a display so that they are not always at the absolute centre. This can allow for the largest possible variety of angles of peripheral visual stimuli to be displayed, e.g. when the central visual stimuli/game is at the far left of the screen, a peripheral visual stimuli can be displayed at the far right of the screen in order get a larger angle of testing than if the central visual stimuli/game had just stayed in the central region.

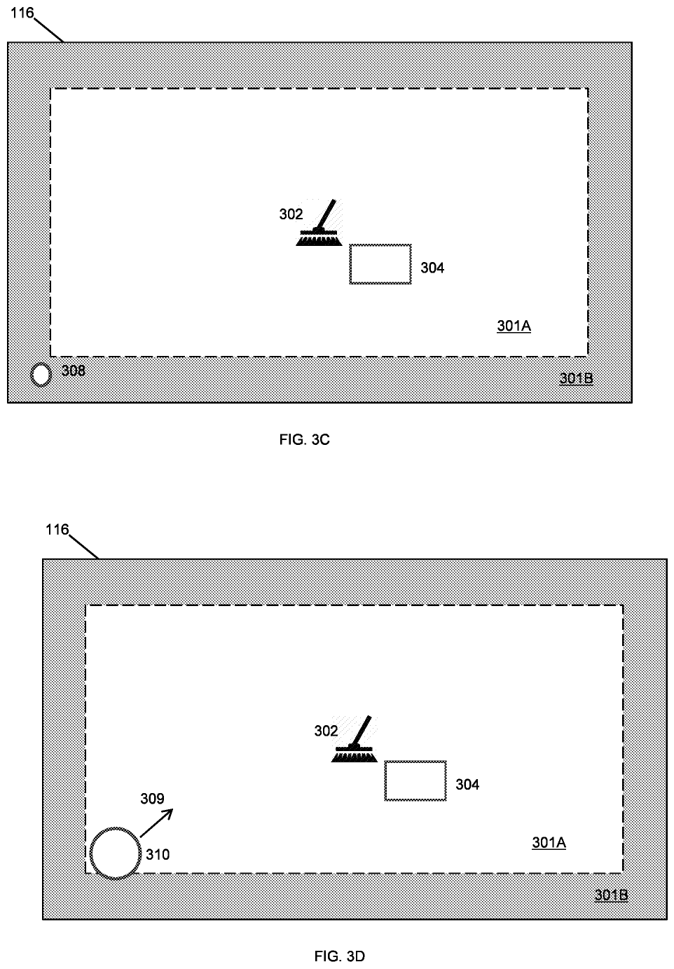

FIG. 3A is a simplified example of a first display 300A generated by the computer 102 and shown on the display device 116. The display is generated by a set of instructions/software stored at least temporarily in the memory 106 of the computer and executed by its processor 104. It will be understood that in other embodiments/cases, some of the steps described herein may be re-ordered, omitted and/or repeated. Additional steps may also be performed. Some steps may be performed concurrently instead of sequentially. It will also be understood that embodiments of the methods described herein can be implemented using any suitable programming language/means and/or data structures.

Some embodiments can comprise displaying a first visual stimulus in a central display region of the display device 116 and displaying a second visual stimulus in a peripheral display region of the display device. An intention of some embodiments is that the user maintains his/her focus on the central display region whilst also being tested whether he/she can see/correctly visually detect the second visual stimulus in the peripheral display region.

Typically, the central display region will be in a substantially central area of the overall display area/screen of the display device 116. The central display region is schematically illustrated by rectangle 301A in the Figure; however, it will be understood that this is for illustration only and it may not be actually displayed during execution of the method. It will also be appreciated that the outline/shape (e.g. it need not be rectangular/square, but could be elliptical or the like) and dimensions of the central display region can vary. In general, the central display region will be centred around a substantially central point of the overall display area/screen of the display device, and will be spaced apart from some/all of the edges of the overall display area/screen. For example, there may be a margin of up to around 20% of the overall display area, between an edge of the central display region and an adjacent edge of the overall display area/screen. In general, the areas may use as much of the screen area as possible in order to get the broadest possible range of angles of the periphery-central target.

Typically, the peripheral display region will comprise substantially peripheral regions of the overall display area/screen of the display device 116. The peripheral display region is schematically illustrated/shaded at 301B in the Figure; however, it will be understood that this is for illustration only and the area may not be actually denoted during execution of the method. Again, it will also be appreciated that the outline/shape and dimensions of the peripheral display region can vary. In general, the peripheral display region will comprise edge regions located away from/excluding the central area of the overall display area/screen of the display device. For example, the peripheral display region may comprise margins of up to around 20% of the overall display area, or corner regions of the whole display area/screen.

In some embodiments, the visual field assessment method may comprise intermittently displaying the first visual stimulus in the central display region 301A. The user may indicate that he/she has seen/visually detected the first visual stimulus by using the user input device 118, e.g. by pressing a button. In some embodiments the processor may determine that the user has correctly visually detected the first visual stimulus if the use of the user input device indicates that the user activated the user input device within a time frame related to the intermittent display of the first visual stimulus, e.g. the user input device is activated within a predetermined time period (e.g. number of microseconds) following the display of the first visual stimulus (or within a predetermined time period after the first visual stimulus has disappeared/stopped being displayed). The first visual stimulus may comprise a first display object being displayed in (or changing to) a certain/predetermined display state, e.g. appearing then disappearing, enlarging, changing colour, changing position, etc.

In some embodiments, the visual field assessment method may further comprise intermittently displaying the second visual stimulus in the peripheral display region 301B. The user may indicate that he/she has seen the second visual stimulus by using the user input device 118. Again, in some embodiments the processor may determine that the user has correctly visually detected the second visual stimulus if the use of the user input device indicates that the user activated the user input device within a time frame related to the intermittent display of the second visual stimulus, e.g. the user input device is activated within a predetermined time period (e.g. number of microseconds) following the display of the second visual stimulus (or within a predetermined time period after the second visual stimulus has disappeared/stopped being displayed). The second visual stimulus may comprise a second display object being displayed in (or changing to) a certain/predetermined display state, e.g. appearing then disappearing, enlarging, changing colour, changing position, etc.

The intermittent displaying of the first and second visual stimuli will normally be based on a particular timing schedule, e.g. a pre-stored schedule and/or an algorithmic or event-driven approach to generating display events. For instance, the first and/or the second visual stimulus may flash on/off.

As mentioned above, some users, particularly children, are often only able to cope with one input button. In order to allow for this they may be instructed (and/or trained in an early “training game”) to respond quickly to the appearance of the first visual stimuli/central targets. The second visual stimuli/peripheral targets may only be displayed during a time window when a central target does not require a response This time window may be adjusted during the gameplay, e.g. based on a user's performance.

The example first display 300A can comprise at least one graphical object that can be notionally controlled by the user using the user input device 118. In the example this comprises a brush/vacuum object 302. The example display can further comprise at least one further graphical object. In the example this comprises a receptacle object 304. The example display can further comprise at least one graphical object that can act as the first visual stimulus. In the example this comprises a “googlie” object 306 that is displayed intermittently. In the example display of FIG. 3A it comprises small disc shaped object that hovers under the brush (a “googlie-in-waiting”). Every so often it changes into a full round circular googlie 307, as shown in FIG. 3B. The user should press the response button when this appears.

To encourage the user to maintain attention and visual focus on the central/main game the method may comprise providing a positive consequence (in terms of the game's rules) for indicating that he/she has correctly seen the first visual stimulus. For example, a user's score may be increased (e.g. +100 points) for each correctly detected googlie 307. Additionally or alternatively, the method may comprise providing a negative consequence for incorrectly indicating seeing, and/or missing, the first visual stimulus. For example, if the user does not press the button when the googlie 307 does appear, the googlie monster will fly up and hit the prince in the face. As a further example, if the user presses the button when there is no first visual stimulus/central googlie 307 then the prince will fall and the user's score will be reduced by 100 points.

Referring to FIG. 3C, during the game, every so often/intermittently, the second visual stimulus, e.g. a peripheral round circle 308 (googlie), will be displayed at a location within the peripheral display region 301B. This can be at different angles and directions with respect to the position of the objects of the central game. The location may be determined by embodiments in a random/pseudo-random manner, retrieved from a store comprising a set of locations, and/or be determined in an algorithmic or event-driven manner, etc. In some embodiments, the position of the central game with the prince may occasionally move around the screen (e.g. after a certain number of central googlies are caught, or after a certain period of time has elapsed) in order to facilitate a maximum variety of display locations of objects of the peripheral game.

When the user sees/visually detects a peripheral googlie 308, he/she should press the response button of the user input device 118. The googlie will then move (“be sucked in”) towards the prince and in some cases it will be shown moving into, and filling up, the receptacle object 304 to indicate successful visual detection. The method may comprise providing a positive consequence (in terms of the game's rules) for indicating that the user has correctly seen the second visual stimulus. For example, for every peripheral/second stimuli correctly detected the user may gain one small star. Once they reach 10 they get a large star. Of course, other/additional rewards, such as increasing the user's score in some other manner, are possible.

In this way the user has maximum incentive to ensure he/she looking at the central game (or else they will have central googlie 306 on their face), but still press the button when they see a peripheral object/googlie 308. The user's gaze isn't unduly drawn towards the periphery as those objects are moving (“being sucked”) inwards/centrally. Thus, selectively displaying a graphical object in the form of the second visual stimuli moving (as an animation) from the peripheral display region towards the central display region based on a determination as to whether the user has correctly visually detected the second visual stimulus (based on their use of the user input device to indicate their visual detection of it) can have the beneficial technical effect of an improved visual field assessment system/method that provides results having improved accuracy.

If the user presses the input button when there is no central or peripheral target requiring a response then they may see an error graphic and/or lose points. Some users usually soon get out of the habit of doing this—it is theroretically possible to positively score by pressing out of being trigger happy, if at the precise time a target appeared, but would be very rare and players would not find this a fruitful gaming strategy.

There can be many precise peripheral locations that are each tested in order (e.g. random or predetermined) until they all have been tested. If one of the second visual stimuli is missed then it may be displayed/tested a second time. The size of each of these has been determined from earlier studies to represent target sizes that should be just visible for children of the relevant age for each location. The locations are normally based on standard literature in the field of vision assessment and can be varied depending on type of visual field defect being looked for, e.g. a neurological disease would be different to eye disease. The sizes may be determined by extensive published studies looking at threshold levels in normal children of different ages.

It has been found that sometimes a user gets too engrossed in the central game and may forget to take the periphery into account. It is therefore desirable to provide some form of negative consequence if they don't pay attention to the periphery. It is beneficial that they do not just lose points and be retested on that location—if they have a genuine field defect then they would just keep losing points and never finish the game, or be disheartened. It is not desirable to make the dot explode or have any excessive peripheral visual impact because they wouldn't see it and nothing should be done during gameplay that might make them stop fixing their gaze on the central game.

Some embodiments can solve this problem by using modified second visual stimuli (called “error dots”). Only after a user has seen at least one “normal” peripheral visual stimulus is this modified process used. A normal peripheral visual stimulus is one that is displayed to test if it can be correctly seen. If it is then it is recorded as shown once and seen for that location, e.g. recorded in an XML file.

If it is not correctly detected then it may be tested again later once. It can then be recorded as tested twice and as correctly seen or not correctly seen second time, e.g. in the XML file. If the user misses a certain number of, e.g. any two consecutive, peripheral visual stimuli then modified second visual stimuli/“error dot” is displayed at one of the last locations where a normal second stimuli was correctly detected by the user. In some embodiments the modified second visual stimuli/error dot is larger than a normal second visual stimulus. The user should therefore be able to easily see it because it is displayed at one of the last locations where a second visual stimulus was previously correctly detected. Of course, the modified second visual stimulus could be visually different to the normal second visual stimulus in some other way, e.g. may be a different shape, a different colour, etc.

Therefore, If the user misses this modified second visual stimuli/error dot, it is not because he has a field defect but more likely it is due to not concentrating on, or complying with, the game/assessment. In some embodiments, an error dot negative feedback cartoon may then activated, which consists of that same larger error dot (310 in FIG. 3D) moving/“flying” in (as denoted by arrow 309) from the periphery towards the central display region to hit the prince in the face. This serves to remind the user regarding the periphery and can re-stimulate them to respond. The dot flying in towards the centre prevents too much attention being drawn away from the centre game. Thus, selectively displaying a graphical object in the form of the modified second visual stimuli moving (as an animation) from the peripheral display region towards the central display region based on a determination as to whether the user has correctly visually detected the second visual stimulus (based on their use of the user input device to indicate their visual detection of it) can, again, have the beneficial technical effect of an improved visual field assessment system/method that provides results having improved accuracy.

Once these steps have been performed the two missed peripheral second stimuli can be tested a further (last) time. Thus, an error dot can be a modified second visual stimulus that is intended to “wake up” a user. It may be bigger in size than a normal second visual stimulus. The number of error dots shown/seen may be displayed at the end of the game (but these don't count on the XML file that is used to generate the visual field map for the child). The XML (or other type of output) can be automatically generated based on whether the normal intended size stimuli is seen or not, whose sizes and locations may be designated before the start of the test from historical/experiment-based databases. Graphs can be created from this data.

To give a detailed example involving the modified second visual stimuli/error dots:
A peripheral/second stimulus is displayed in location A of the peripheral display region
The user sees the peripheral stimulus and presses the button (resulting in it being “vacuumed up”)
A peripheral/second stimulus is presented in location B of the peripheral display region
The user does not correctly detect it by pressing the button in time
A peripheral/second stimulus is presented in location C of the peripheral display region
The user does not correctly detect it by pressing the button in time
A modified peripheral/second stimulus is re-displayed at the location A as “the error dot” 310. This modified stimulus is larger (e.g. 2 sizes large). As the user has previously seen a stimuli in this location A, he/she should easily be able to see a larger stimuli in the same location. If it is missed then it could be due to lack of concentration as opposed to loss of field.
If the user sees the modified second stimulus/error dot, the method continues as normal
If the user misses the modified second stimulus/error dot then it will ‘fly in’ 309 to hit the prince in an attempt to regain the user's concentration
Whether the user sees the modified second stimulus/error dot or not, the missed second stimuli are immediately re-tested, counting as their second time of testing.
A user must miss two consecutive stimuli for the modified second stimulus/error dot to be shown. If a user misses one stimuli but then captures the next stimuli shown then the error dot is not shown and the missed stimuli is simply re-displayed later in the game for a second time.
An error dot will only be displayed if the child has correctly detected at least one second stimuli during the method/game.

An examiner supervising the user of the system 100 can see the total number of error dots presented and missed at the end of the game. It there are a large number of missed error dots then it can be deduced that the field is not reliable, which can be considered use for the error dots. Some embodiments can generate an output indicating the user's performance, which can be output in various ways.

In some embodiments reliability indices may be displayed at the very end of a game, when all peripheral visual stimuli have been tested. Embodiments may produce an output indicating all the locations where the visual stimuli were correctly seen/detected. To determine the reliability/validity of the testing, the output may also include additional information, such as: number of error dots presented; number of error dots missed (this is important as users with defects may miss several peripheral stimuli in a row due to their defect); number of central visual stimuli/googlies missed (to help assess possible loss of central fixation); number of times the prince fell down (may indicate the user was ‘trigger happy’).

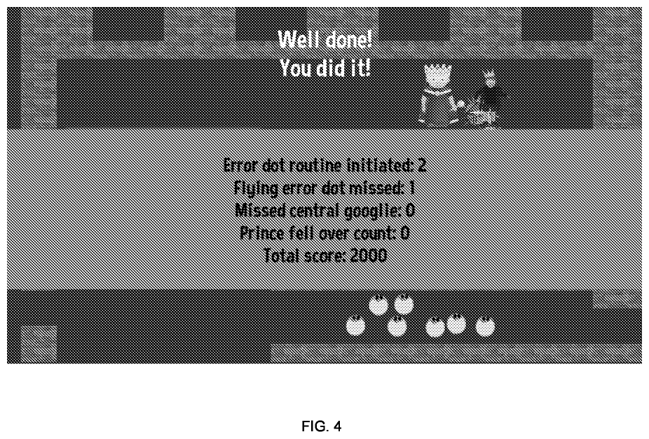

FIG. 4 shows an example output being displayed on the display device 116.

Thus, the error dot feature can be considered to have multiple functions/benefits, including:
1. To encourage a user to pay attention to the periphery in general by reminding them about it
2. To make sure gameplay continues even if the user has a large visual field defect by often forcing some peripheral dots to appear in those places where the user has demonstrated he/she can see
3. To re-test areas that might have been missed because of inattention
4. To provide an indication of the overall reliability of the test score at the end

Some embodiments of the system 100 may use the optional eye/gaze tracking device 119.

Embodiments may use eye tracking technology purely to establish if gaze is directed as expected at the central game. Only when tracking confirms this will peripheral targets in the peripheral display region be shown. This is better than existing systems which use eye tracking to determine if peripheral objects are seen or not seen—that gets tiring for users and the accuracy is poor.

Embodiments are therefore simpler and yet just as effective and can keep the user engaged with a game.

To maintain the interest of the user/child, some embodiments may provide multiple levels of the game. For instance, 10 visual stimuli are presented in one level, increasing to 20 in a subsequent level. For example, in some cases in levels 2 and 3 if the child correctly detects several second visual stimuli in a row then the game may speed up. The game may slow back down if the child missed the dots again. In some embodiments, in level 3 a cat is sometimes presented in the centre instead of a central googlie 307. They shouldn't press the button if this happens—If they press the button then the cat is hit. In some embodiments the game may have a version with a slightly increased response time, and levels 2 and 3 may not speed up as much for children who struggle. A user may be offered a break between levels and the game may be paused by an operator at any time.

To help make sure users understand how to use the system 100, some embodiments may provide training levels to be used before starting the game proper. These can begin with just the central game and no time requirements to press the button in time and no negative consequences. The training level may include a level where the central googlie monster and outside stimuli will not disappear until the user presses the button. This level can be used to ensure the user understands when they need to press the button.

In one trial, patients with neurological developmental delay were identified and assessed for suitability for testing the system. Exclusions included visual activity wore than 6/24 and physical conditions that may affect ability to use the system, ones identified as being unsuitable due to severe cognitive impairment, and so on. Clinical histories of each of the consenting patients were recorded, which included their diagnosis, visual acuity and results from any previous visual field examination where possible. Children and their guardians were then asked about their attitudes towards computer games and their past experiences of visual field testing. In one study, 15 patients (7 girls and 8 boys) consented to take part (mean age 7.5 years, range 4-14 years). Two of these (aged 6 and 7) were unable to progress past the training levels, both had a diagnosis of global development delay. In the remaining 13 patients, 6 were able to complete to test for both eyes and 7 for one eye. In the 7 children in which it was not possible to obtain bilateral visual fields, 4 were due to low attention span, 1 was due to fatigue, 1 was due to time constraints and 1 was due to poor visual acuity in the second eye. Of the 4 children who were unable to complete a second visual field test due to low attention span, 2 had ADHD and 2 had cognitive impairment.

Of the 15 children tested, 5 had neurofibromatosis, 1 had CNS tumours, 2 did not yet have a formal diagnosis, 1 had optic atrophy due to demyelination, 1 had a Chiari 1 malformation, 1 had Niemann-Pick disease and 1 had sagittal craniosynotosis.

In the study, each patient was discussed individually and reliability indices and comparisons with Humphreys visual fields were done where possible, e.g. using the Excel files that were automatically generated by the system. Feedback about the system was largely positive; for example, 100% of the children that had past experience of formal visual testing reported preferring the system due to it being more enjoyable and maintaining concentration on it for prolonged periods of time being much more fun, especially those with ADHD for whom a conventional visual field test would have proved impossible.

The results of the study demonstrated that embodiments can be successfully used to measure visual field in children with neurodevelopmental delay. Even children as young as 4 and with ADHD and cognitive impairment were able to complete the test. This can prove invaluable to this group of children in whom testing is generally limited to confrontation testing in which only gross defects are detected. Embodiments can also be particularly beneficial in children with neurofibromatosis type 1 in whom the majority of optic gliomas are diagnosed prior to the age of 6 years.

Results from the tests were comparable with Humphrey's visual field tests. Thus, embodiments can contribute considerably to the development and evaluation of vision tests in developmentally delayed children in particular. Research shows that parents of developmentally delayed children report greater unmet healthcare needs than parents of those developmentally normal children. It was also found that the majority of children who had bilateral tests performed the tests not only faster in the second eye, but with better reliability indices. An implication of these findings is that a version of the system implemented on a tablet computer or the like can be used for a child to practice at home, which may increase reliability and reduce appointment time during formal assessment.

Although preferred embodiments have been shown and described, it will be appreciated by those skilled in the art that various changes and modifications might be made without departing from the scope of the invention, as defined in the appended claims and as described above.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims and drawings), and/or all of the steps of any method or process so disclosed, may be combined in any combination, except combinations where at most some of such features and/or steps are mutually exclusive.

Each feature disclosed in this specification (including any accompanying claims, and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims and drawings), or to any novel one, or any novel combination, of the steps of any method or process so disclosed.

1. A visual field assessment system comprising:
a display device having a display area;
a user input device, and
a processor configured to:
intermittently display a first visual stimulus in a central display region of the display area;
intermittently display a second visual stimulus in a peripheral display region of the display area;
determine whether the user has correctly visually detected the second visual stimulus based on use of the user input device, and
selectively display a graphical object moving from the peripheral display region towards the central display region based on the determination.

2. The system according to claim 1, wherein the processor is configured to determine that the user has correctly visually detected the second visual stimulus if the user activated the user input device within a time frame related to the intermittent display of the second visual stimulus following the display of the second visual stimulus, or within a predetermined time period after the second visual stimulus has disappeared/stopped being displayed.

3. The system according to claim 1, wherein the processor is configured to selectively display the graphical object moving from the peripheral display region towards the central display region as an animated display.

4. The system according to claim 3, wherein the second visual stimulus comprises intermittently displaying the graphical object in the peripheral display region.

5. The system according to claim 1, wherein the processor is configured to intermittently display a plurality of the second visual stimuli at different locations within the peripheral display region.

6. The system according to claim 5, wherein the processor is configured to: determine that the user has correctly visually detected at least one of the plurality of second visual stimuli based on use of the user input device;
intermittently display a first further one of the plurality of second visual stimuli at a first location within the peripheral display region;
determine whether the user has correctly visually detected the first further one of the plurality of second visual stimuli based on use of the user input device;
intermittently display a second further one of the plurality of second visual stimuli at a second location within the peripheral display region;
determine whether the user has correctly visually detected the second further one of the plurality of second visual stimuli based on use of the user input device, and
if the processor determines that the user has not correctly visually detected the first further one and the second further one of the plurality of second visual stimuli then the processor is configured to display a modified second visual stimulus.

7. The system according to claim 6, wherein the modified second visual stimulus is visually different to the second visual stimulus, e.g. a different size.

8. The system according to claim 6, wherein the modified second visual stimulus is displayed at the first location of the peripheral display region.

9. The system according to claim 6, wherein the processor is configured to determine whether the user has correctly visually detected the modified second visual stimulus based on use of the user input device and if the processor determines that the user has not correctly visually detected the modified second visual stimuli then the processor is configured to display the modified second visual stimuli moving from the peripheral display region towards the central display region.

10. The system according to claim 6, wherein the processor is configured to subsequently re-display the second further one of the plurality of second visual stimuli at the second location within the peripheral display region, and determine whether the user has correctly visually detected the second further one of the plurality of second visual stimuli based on use of the user input device.

11. The system according to claim 1, further comprising an eye/gaze tracking device, wherein the processor is configured to use the eye/gaze tracking device to determine if a gaze of the user is directed at the central display region during the intermittent display of the second visual stimulus.

12. The system according to claim 1, wherein the processor is configured to intermittently display a plurality of the first visual stimuli at a plurality of different locations within the central display region.

13. The system according to claim 1, wherein the processor is configured to generate an output indicating a performance of the user in visually detecting at least the second visual stimuli.

14. The system according to claim 13, wherein the output comprises at least one of: a number of modified second visual stimuli displayed; a number of modified second visual stimuli missed by the user; a number of first visual stimuli missed.

15. The system according to claim 1, wherein the second visual stimulus is displayed during a time window when the first visual stimulus is not displayed, wherein the time window is adjusted during use, e.g. based on a user's performance.

16. The system according to claim 1, wherein the processor is configured to provide a positive consequence if the user has correctly visually detected the first visual stimulus and/or the second visual stimulus, and wherein the processor is configured to provide a negative consequence if the user has not correctly visually detected the first visual stimulus and/or the second visual stimulus.

17. The system according to claim 1, further comprising a panel that, in use, is positioned/positionable between the user and at least one of the display device and the user input device.

18. The system according to claim 17, wherein the panel comprises a surface of a housing containing at least one of the display device and the user input device.

19. The system according to claim 1, wherein the processor is configured to generate a display in the central display region such that a gaze of the user is focused/maintained on the central display region even whilst the second visual stimulus is intermittently displayed in the peripheral display region.

20. A computer-implemented visual field assessment method comprising:
intermittently displaying a first visual stimulus in a central display region of a display area of a display device;
intermittently displaying a second visual stimulus in a peripheral display region of the display area;
determining whether a user has correctly visually detected the second visual stimulus based on use of a user input device, and
selectively displaying a graphical object moving from the peripheral display region towards the central display region based on the determination.

21. A computer readable medium storing a computer program to operate a visual field assessment method according to claim 20.
